(19) United States
(12) Patent Application Publication
(10) Pub. No.: US 2011/0048144 A1
(43) Pub. Date: Mar. 3, 2011

(54) METHOD FOR MEASURING THE DYNAMICS OF ROOT DEVELOPMENT AND APPARATUS FOR CARRYING OUT SAID METHOD

(75) Inventors: Peter ZELEZNIK, Grize (SI); Mitja FERLAN, Preserje (SI)

(73) Assignee: GOZDARSKI INSTITUT SLOVENIJE, Ljubljana (SI)

(21) Appl. No.: 12/862,206

(22) Filed: Aug. 24, 2010

(30) Foreign Application Priority Data
Aug. 28, 2009 (SI) SI P-200900233

(51) Int. Cl. G01N 33/24

(52) U.S. Cl. 73/866

(57) ABSTRACT

The present invention relates to the measurement of growth and decay of roots, especially fine roots of green plants using the probe to be inserted into the soil being investigated. According to the invention, an insertion (6) is arranged in a transparent research tube (2), whereby a plurality of sensors (10), a data logger (11) and/or a power supply (12) are arranged in said insertion (6).

### CROSS-REFERENCE TO RELATED APPLICATIONS

This application claims the benefit of priority from Slovenian Patent Application P-200900233, filed Aug. 28, 2009.

### BACKGROUND OF THE INVENTION

1. Field of the Invention

The present invention refers to a method of measuring the dynamics of root development by means of a probe inserted into the soil being investigated, and to an apparatus for carrying out said method.

2. Description of the Related Art

A method and an apparatus of the aforementioned kind are known from a U.S. Pat. No. 4,445,788. Known method is carried out by means of a probe comprising a plurality of sensors, said probe being inserted in the soil being investigated, and senses a plurality of soil characteristics at different locations. An apparatus for carrying out said known method comprises a hollow shell and at least two sensors for sensing soil characteristics, a display and a device for transfer of data from said sensors to said display. At least one of said sensors is of a kind to sense temperature. It is a drawback of said known solution that the temperature conditions in said hollow shell differs substantially from those in the surrounding soil having a result that collected results do not correspond to the real situation. In addition, said probe is subjected to the water condensate resulting in a necessity to clean said probe leading to unwanted movements thereof.

Furthermore, an apparatus is known (a so-called minirhizotron observation tube with the associated equipment) of a company Bartz Technology Corporation, by means of which the dynamics of root development is measured. Also with this embodiment, a significant drawback is represented by the fact that the temperature conditions in said apparatus differs substantially from those in the surrounding soil resulting in a non-compliance with the real situation. In addition, said probe is also subjected to the water condensate resulting in a necessity to clean said probe leading to unwanted movements thereof.

### BRIEF SUMMARY OF THE INVENTION

It is the object of the presented invention to create a method for measuring the dynamics of root development, which will remedy drawbacks of a known solution.

A further object of the invention is to create an apparatus for carrying out said method thus enabling a soil temperature profile in a probe as real as possible.

According to the present invention, the object as set above is solved in a manner that a transparent research tube (also called minirhizotron) is installed at the research site, an insertion being placed inside said research tube, said insertion consisting of a carrier pipe and a radially extended projection. After the expiration of required time, a user reads the measured values stored in a data logger where they have been transmitted by sensors, arranged in said carrier pipe of said insertion. Afterwards, the user removes said insertion from said transparent research tube and installs a camera into said transparent research tube. This action is followed by a picture taking session in said transparent research tube and re-insertion of said insertion into said transparent research tube.

### BRIEF DESCRIPTION OF THE FIGURES

An apparatus for carrying out a method according to the invention is further described in detail on the basis of the preferred embodiment and with reference to the accompanying drawings, where

FIG. 1 shows a partial longitudinal section of an apparatus for carrying out a method according to the invention,

FIG. 2 shows a partial longitudinal section of an apparatus for carrying out a method according to invention in a three dimensional view.

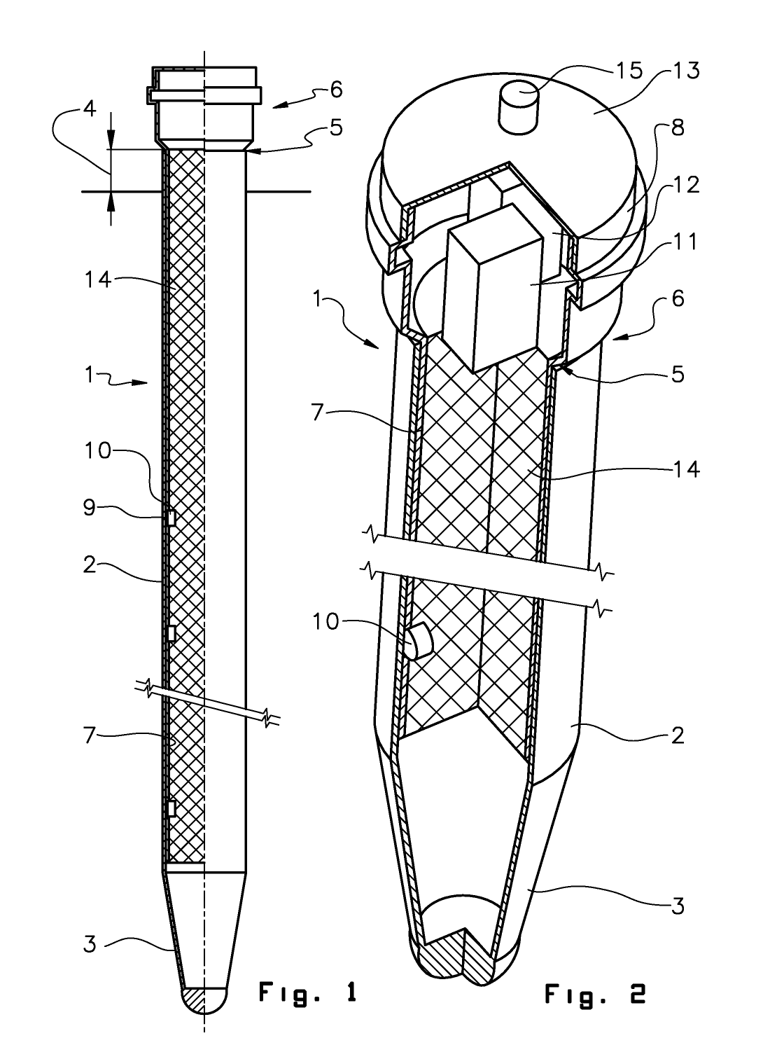

### DETAILED DESCRIPTION OF THE INVENTION

An apparatus according to the invention for measuring dynamics of root development by means of a probe 1 inserted into the investigated soil, consists of a transparent research tube 2, which is at the end thereof intended to penetrate into the soil closed with a tip 3 that facilitates penetration of said tube 2 in a ready-made hole in the ground. Said research tube 2 is inserted into the soil to be investigated, in a manner that a smaller section 4 thereof protrudes above the surface of said soil, so that a user can easily access an entry 5 of said tube 2. Afterwards an insertion 6 is placed through said entry 5 into said research tube 2, said insertion 6 being formed in a manner that the outer diameter thereof fits with a loose fit to the internal diameter of said research tube 2, so that it can still be pulled out without requiring too much effort or special equipment.

Said insertion 6 having essentially the same length as the research tube 2, comprises a carrier pipe 7, a radially extended projection 8 being attached thereto at the end protruding from the research pipe 2, said projection 8 seats on said entry of said tube 2, thus preventing said insertion to slip too far into said research tube 2 when inserted therein. Said carrier pipe 7 is formed in the longitudinal direction thereof with plurality of radially extended through holes 9, temperature and/or similar sensors 10 being arranged therein. Each sensor 10 is connected to a data logger 11, comprising its own power supply, and/or to a power supply 12 being preferably arranged within said radially extended projection 8. Said projection 8 is efficiently closed in a manner known per se by means of a removable lid 13, thus preventing entry of moisture and/or other impurities in the interior of said carrier pipe 7 and said projection 8, respectively. The interior of said carrier pipe 7 is filled, from the area of the connection with radially extended projection 8 to the free end of said pipe 7, arranged in said research tube 2, with a heat insulating filler 14, which tightly fits to said carrier pipe 7 and simultaneously fixes said sensors 10. Said lid 13 is optionally formed with communication port 15 being associated with said data logger 11 and represents a connection point with optional device not shown, such as portable computer, to which either occasionally or continuously data are sent being previously transmitted by said sensors 10 to said data logger 11.

According to the invention, it is preferably provided for that said power supply 12 is located in said extended projection 8 of carrier pipe 7. However, it is also possible for said power supply to be arranged outside said carrier pipe 7, even at a remote location, and is in a manner known per se connected with said data logger 11 and/or sensors 10.

Measuring the dynamics of root development by means of said probe inserted into the soil being investigated, is carried out by means of the method according to the invention in a manner that the above described probe 1 according to the invention is inserted into the soil being investigated, whereby at first the transparent research tube 2 is inserted into the soil. Afterwards, said insertion 6 is placed into said research tube 2, by means of said insertion 6 said research tube 2 has been completely and tightly closed.

When the user reaches the measurement point, being represented by each probe 1, the data are transferred at first from said data logger 11, using for example a portable computer. Afterwards, that the user removes said inserted insertion 6 out of said research tube 2 and puts thereon a camera known per se and not shown, and at different depths of said tube 2 takes the required number of relevant images of roots.

1. A method of measuring growth and decay of roots, particularly fine roots of green plants using the probe, inserted into the soil being investigated, characterized in that it comprises the following steps:
a) installing a transparent research tube (2) at the location of a measurement,
b) entering insertion (6) in said research tube (2),
c) reading, after the required time, measured values from data logger (11)
d) extracting insertion (6) from said transparent research tube (2),
e) installing camera in said transparent research tube (2),
f) taking set of photos in said transparent research tube (2), and
g) re-entering insertion (6) in said transparent research tube (2).

2. A method according to claim 1, characterized in that said measured values according to step c) are either temperature and/or similar.

3. An apparatus for carrying out said method according to claim 1, comprising a probe (1), that can be inserted into the soil being investigated, characterized in that it comprises an insertion (6) arranged in a transparent research tube (2), whereby a plurality of sensors (10), a data logger (11) and/or a power supply (12) are arranged in said insertion (6).

4. An apparatus according to claim 3, characterized in that insertion (6) comprises a carrier pipe (7), a radially extended projection (8) being attached thereto at the end thereof projecting out of the research tube (2), and a heat insulating filler (14) which tightly fills the interior of said carrier pipe (7) and tightly fits thereto, and whereby fixes said sensors (10).

5. An apparatus according to claim 3, characterized in that said carrier pipe (7) is formed at least on a section of the length thereof with a plurality of radially extending through holes (9) in which sensors (10) are arranged.

6. An apparatus according to claim 5, characterized in that said sensors (10) are temperature and/or similar sensors.

7. An apparatus according to claim 3, characterized in that each sensor (10) is connected with the data logger (11) and/or power supply (12), which are preferably located within said radially extended projection (8) of the carrier pipe (7).

8. An apparatus according to claim 3, characterized in that said projection (8) is closed by means of a removable lid (13), a communication port (15) being attached thereto.

9. An apparatus for carrying out said method according to claim 2, comprising a probe (1), that can be inserted into the soil being investigated, characterized in that it comprises an insertion (6) arranged in a transparent research tube (2), whereby a plurality of sensors (10), a data logger (11) and/or a power supply (12) are arranged in said insertion (6).

10. An apparatus according to claim 4, characterized in that said carrier pipe (7) is formed at least on a section of the length thereof with a plurality of radially extending through holes (9) in which sensors (10) are arranged.

11. An apparatus according to claim 4, characterized in that each sensor (10) is connected with the data logger (11) and/or power supply (12), which are preferably located within said radially extended projection (8) of the carrier pipe (7).

12. An apparatus according to claim 5, characterized in that each sensor (10) is connected with the data logger (11) and/or power supply (12), which are preferably located within said radially extended projection (8) of the carrier pipe (7).

13. An apparatus according to claim 6, characterized in that each sensor (10) is connected with the data logger (11) and/or power supply (12), which are preferably located within said radially extended projection (8) of the carrier pipe (7).

14. An apparatus according to claim 4, characterized in that said projection (8) is closed by means of a removable lid (13), a communication port (15) being attached thereto.

15. An apparatus according to claim 5, characterized in that said projection (8) is closed by means of a removable lid (13), a communication port (15) being attached thereto.

16. An apparatus according to claim 6, characterized in that said projection (8) is closed by means of a removable lid (13), a communication port (15) being attached thereto.

17. An apparatus according to claim 7, characterized in that said projection (8) is closed by means of a removable lid (13), a communication port (15) being attached thereto.
